(19) United States
(12) Patent Application Publication
(10) Pub. No.: US 2022/0409114 A1
(43) Pub. Date: Dec. 29, 2022

(54) SYSTEM AND METHOD FOR PERSONALIZED KIDNEY EVALUATION, DIAGNOSIS AND THERAPY RECOMMENDATION

(71) Applicant: QUADRUS MEDICAL TECHNOLOGIES, INC., White Planins, NY (US)

(72) Inventors: Nicolas Wadih Chbat, Ardsley, NY (US); Caitlyn Marie Chiofolo, New Hyde Park, NY (US)

(21) Appl. No.: 17/761,399

(22) PCT Filed: Sep. 17, 2020
(86) PCT No.: PCT/US2020/051257

§ 371 Date: Mar. 17, 2022

Related U.S. Application Data
(60) Provisional application No. 62/901,478, filed on Sep. 17, 2019.

(51) Int. Cl. A61B 5/20; G06F 30/28

(52) U.S. Cl. CPC A61B 5/201; G06F 30/28; G06F 2111/10

(57) ABSTRACT

A system for evaluating a kidney includes a processing device including an input module configured to acquire patient information, the patient information including at least one of demographic data, diagnostic data, physiological data and intervention data. The processing device also includes an evaluation module, which is configured to input patient class data to an initial kidney model, the initial kidney model configured to simulate a physiological response of a kidney and configured to simulate fluid and solute transport through one or more spatial locations of the kidney. The evaluation module is also configured to input patient data corresponding to an individual patient and calculating a model response, and adjust at least one parameter of the initial kidney model based on a comparison of the patient data and the model response to personalize the initial kidney model for the individual patient.

### CROSS REFERENCE TO RELATED APPLICATIONS

This application is a National Stage of International Application No. PCT/US2020/51257 filed on Sep. 17, 2020, which claims the benefit of U.S. Provisional Application No. 62/901,478, filed Sep. 17, 2019, which are incorporated herein by reference in their entirety.

### SUMMARY

An embodiment of a system for evaluating a kidney includes a processing device including an input module configured to acquire patient information, the patient information including at least one of demographic data, diagnostic data, physiological data and intervention data. The processing device also includes an evaluation module, which is configured to input patient class data to an initial kidney model, the initial kidney model configured to simulate a physiological response of a kidney and configured to simulate fluid and solute transport through one or more spatial locations of the kidney. The evaluation module is also configured to input patient data corresponding to an individual patient and calculating a model response, and adjust at least one parameter of the initial kidney model based on a comparison of the patient data and the model response to personalize the initial kidney model for the individual patient.

An embodiment of a method of evaluating a kidney includes acquiring patient information at an input module, the patient information including at least one of demographic data, diagnostic data, physiological data and intervention data, and inputting patient class data to an initial kidney model, the initial kidney model configured to simulate physiological responses of a kidney, the initial kidney model configured to simulate fluid and solute transport through one or more spatial locations of the kidney. The method also includes inputting patient data corresponding to an individual patient and calculating a model response, adjusting at least one parameter of the initial kidney model based on a comparison of the patient data and the model response to personalize the initial kidney model for the individual patient.

### BACKGROUND

The present invention generally relates to assessment, diagnosis and evaluation of organ health. More specifically, the present invention relates to generating and utilizing a mathematical model of an organ, such as a kidney, and personalizing the model to a class of patients and/or individual patients.

Kidney conditions, such as acute kidney injuries, affect a large number of patients globally. Diagnosis and prediction of kidney conditions is difficult and is affected by a large number of variables. Further, the underlying mechanism of disease pathology is often needed to understand how to properly prevent, intervene, or manage renal damage and/or disease. Thus, it can be challenging for physicians to effectively diagnose and treat kidney conditions.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a computer system configured to perform aspects of embodiments of the present invention;

FIG. 2 depicts aspects of a kidney and illustrates kidney functions simulated by a mathematical model according to an embodiment;

FIG. 3 is a flow diagram illustrating kidney spatial locations and functions that could be simulated by a dynamic kidney model such as the one of FIG. 2;

FIG. 4 is a block diagram that depicts a mathematical model of a kidney and a method of personalizing the model, and a method for performing what-if clinical scenarios;

FIG. 5 depicts examples of interventions, model inputs and outputs utilized in the method of FIG. 4; and

FIG. 6 is a functional block diagram depicting aspects of a method of evaluating kidney function and generating treatment recommendations based on a mathematical model of a kidney.

### DETAILED DESCRIPTION

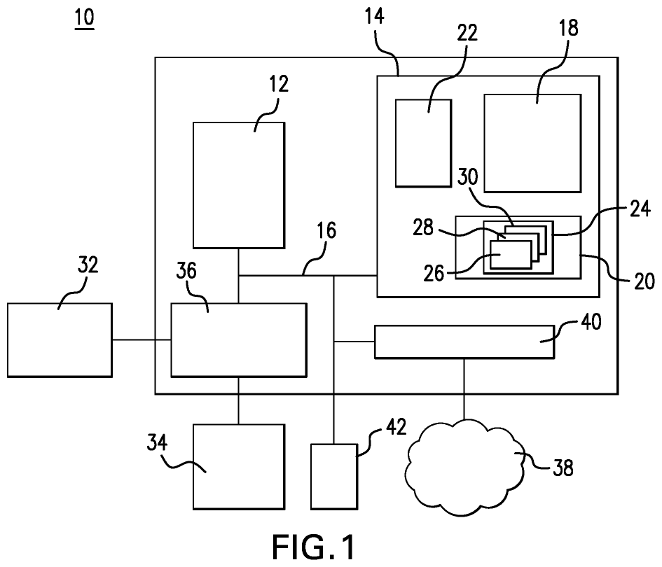

FIG. 1 shows a computer system 10 configured to perform aspects of physiology-driven organ simulation using a mathematical model of an organ, such as a kidney. The computer system 10 may also evaluate kidney function based on the model, diagnose diseases or conditions, and/or recommend treatments to address such diseases or conditions. The model represents the physiology of a kidney or other organ. In one embodiment, the model simulates organ structure at various spatial locations and simulates fluid, solute and ion transport through and between spatial locations.

The model (a single model or multiple may include one or more mathematical equations that relate to physiology. The models may relate to a kidney and/or represent one or more spatial locations of a kidney, and may also relate to transport phenomena of fluid/mass conservation. The model may include differential or time-based equations (e.g., for forecasting), but the model is not so limited. For example, the model may include algebraic equations and/or constraint equations.

In one embodiment, the model includes one or more differential equations for each spatial location. The differential equations represent dynamic solute, ion and fluid transport (i.e., flow as a function of time). Each differential equation includes parameters and/or initial conditions of variables that can be adjusted to tune the model. A “variable” is any quantity representing the state of a system via an equation that represents organ physiology and response, and can potentially be measured (e.g. concentration, pressure, flow, volume).

For example, the model is tuned by adjusting one or more coefficients that multiply a respective variable. Such coefficients are referred to herein as “parameters.” Parameters generally represent physical property or geometry of the system or of the specific node (spatial location) of a system. The model may also incorporate other equations, such as algebraic equations. Generally, physiological or physical properties of a kidney or other organ are used to define various parameters. Parameters (coefficients) are tuned to fit model estimated/simulated variables to actual/measured variables (from the patient/kidney). Although embodiments are described herein in conjunction with simulating a kidney or renal system, they can be utilized with other organs and organ systems.

In one embodiment, the model can be tuned or adjusted to simulate kidney structure and function for a class of patients and/or for an individual patient. The model may be tuned, for example, by acquiring variable and intervention data indicating actual kidney responses of patients to interventions (e.g., introduction of fluid and/or nutrients). Digital representations of the interventions are input to the model to generate simulated kidney responses (a “model response”). The actual kidney responses are compared to the model responses to calculate an error therebetween. The model may then be tuned (e.g., by adjusting one or more parameters) to reduce the error. Such tuning can be performed iteratively as new kidney response data is acquired.

Embodiments described herein present a number of advantages and technical effects. The model and associated methods allow for quick kidney evaluation and diagnosis, so that therapeutic actions can be performed in a timely manner. Acute illnesses of the kidneys can develop relatively fast (e.g., a few days, hours, or minutes) in an ICU (Intensive Care Unit) setting. If not detected in a timely manner, such illnesses can have devastating effects on the kidneys. Embodiments described herein allow for timely detection (or prediction in advance) of a kidney disease or condition, so that a negative renal spiral can be avoided and in many cases kidneys can be saved.

In addition, embodiments described herein provide kidney models that represent the functioning of a kidney in a dynamic state only, in contrast to other methods that simulate steady state. In addition, the model can be fine-tuned to personalize the model for an individual patient. A significant benefit of having a model fine-tuned to a patient is that simulations of therapies can be run on the model instead of on the patient, so that diagnoses can be made without invasive procedures or causing harm or discomfort to the patient. Based on the response of the model to simulated therapies, more informed decisions can be made as to whether or not to attempt the therapy on the actual patient.

Components of the computer system 10 include one or more processors or processing units 12, a system memory 14, and a bus 16 that couples various system components including the system memory 14 to the one or more processing units 12. The bus 16 represents one or more of any of several types of bus structures, including a memory bus or memory controller, a peripheral bus, an accelerated graphics port, and a processor or local bus using any of a variety of bus architectures. The system memory 14 may include a variety of computer system readable media. Such media can be any available media that is accessible by the one or more processing units 12, and includes both volatile and non-volatile media, removable and non-removable media.

For example, the system memory 14 includes a storage system 18 for reading from and writing to a non-removable, non-volatile memory 20 (e.g., a hard drive). The system memory 14 may also include volatile memory 22, such as random access memory (RAM) and/or cache memory. The computer system 10 can further include other removable/non-removable, volatile/non-volatile computer system storage media.

As will be further depicted and described below, system memory 14 can include at least one program product having a set (e.g., at least one) of program modules that are configured to carry out the functions of embodiments of the invention.

For example, the system memory 14 stores a program/utility 24, having a set (at least one) of program modules. The program/utility 24 may be an operating system, one or more application programs, other program modules, and program data. The program modules generally carry out the functions and/or methodologies of embodiments of the invention as described herein.

For example, the program modules include an input module 26 configured to acquire data such as patient data that can be used to generate, adjust and/or personalize an organ system model such as a kidney model. The program modules can also include an evaluation module 28 configured to simulate kidney (or other organ) function using an organ system model, and an output module 30 configured to output information such as simulation or modeling results and/or recommendations generated based on the simulation.

The one or more processing units 12 can also communicate with one or more external devices 32 such as a keyboard, a pointing device, a display, and/or any devices (e.g., network card, modem, etc.) that enable the one or more processing units 12 to communicate with one or more other computing devices. In addition, the one or more processing units 12 can communicate with an external storage device such as a database 34. Such communication can occur via Input/Output (I/O) interfaces 36.

The one or more processing units 12 can also communicate with one or more networks 38 such as a local area network (LAN), a general wide area network (WAN), and/or a public network (e.g., the Internet) via network adapter 40. The processing units 12 can also communicate wirelessly via, for example, a Bluetooth connection 42 or the like. It should be understood that although not shown, other hardware and/or software components could be used in conjunction with the computing system 10. Examples, include, but are not limited to: microcode, device drivers, redundant processing units, external disk drive arrays, RAID systems, tape drives, and data archival storage systems, etc.

The computer system 10 and/or other processing device or system is configured to generate and/or utilize a mathematical model of an organ system. The mathematical model (organ system model) simulates one or more physiological variables or indicators of the organ system, such as fluid pressures and flows, solute concentrations or flows, transport of fluids, responses to interventions and others.

Examples of organ systems include renal systems (e.g., an individual nephron, an individual kidney and/or additional components of the kidney system), respiratory systems (e.g., lungs), circulatory systems (e.g., the heart and/or blood vessels and/or lymph vessels), nervous systems (e.g. central nervous system, autonomic nervous system that includes chemoreflex (with central and/or peripheral chemoreceptors), enteric nervous system, baroreflex), and digestive systems (e.g., stomach, intestines and/or pancreas).

The organ system model may be a model of a kidney and/or other parts of a renal system. In one embodiment, the model simulates or represents the physiology of a kidney, adhering to the kidney structure and solute transport dynamics. In this embodiment, the organ system model is referred to as a “kidney model” or a “virtual kidney.” It is to be understood that discussions of a kidney model are not intended to limit embodiments described herein to any specific organ or organ system.

In another embodiment, the kidney model can be one or more spatial locations of the kidney and/or connected/related organs or organ systems.

The kidney is a quiet and vital organ. The kidney is quiet relative to its noisier neighbors (the heart and lungs) due to heart beats and respiratory breaths; and is vital since it filters the blood which supplies the body's organs with nutrients. If healthy or functioning properly, the kidney removes excess nutrients and harmful waste products from blood through urine and restores or accepts necessary nutrients into the blood stream. This filtration is accomplished chemically and mechanically via a series of wet transport phenomena (diffusion, reaction, bulk motion, etc.) of nutrients that are solute ions and molecules (generally referred to as “solutes”). The paths that the solutes take is via tubes and tubules that start at the aorta, then onto the renal arteries, and end at the collecting ducts and onto the bladder. The path along the nephron tubules is termed the “axial path.” Transport paths also exist in the transverse direction, i.e. from the tubules into the outer/inner medulla and cortex, and vice versa.

The kidney model mimics the structure and physiology of a kidney to understand the functioning and malfunctioning of the kidney. As discussed further below, the kidney model and associated methods and systems can provide a model-based prognosis and/or therapy recommendation.

The kidney model may be a generic model that represents the structure and function of a generalized kidney. For example, an initial version of the kidney model can be generated using general knowledge of kidney physiology and function. The initial model can be adjusted using actual patient data to result in a version of the kidney model the represents a kidney for a selected class of patients that have similar characteristics, such as kidney functions, responses, diseases and/or malfunctions. In addition, the kidney model can be customized or personalized to represent a kidney of an individual patient, which can be used to diagnose and/or recommend treatments.

The kidney model mimics a kidney by representing or simulating fluid and solute transport dynamically (in time) at different locations in the kidneys. For example, the kidney model can be used to simulate how sodium diffuses, gets carried with the bulk motion of the blood through the kidney, and moves via transporters at various spatial locations (e.g., at the epithelial cell of the proximal convoluted tubules, and/or at the inter-space of the proximal straight tubules).

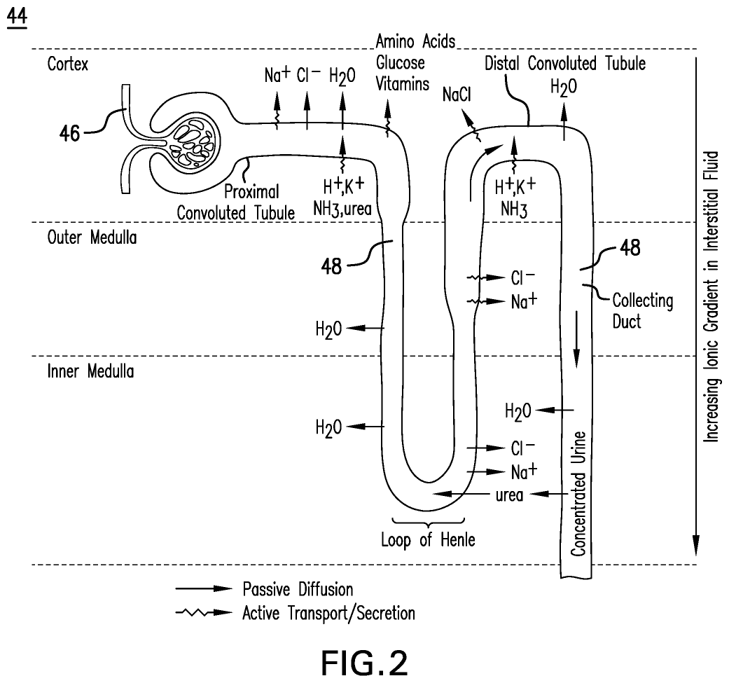

FIG. 2 illustrates various components of a kidney, and illustrates spatial locations, structures and functions (e.g., transport of fluids and solutes through the kidney) that can be simulated using the kidney model. For example, the kidney model simulates fluid and solute transport through blood vessels, nephrons and/or other spatial locations and structures of the kidney.

FIG. 2 depicts a nephron 44 of a kidney. The kidney is made up of numerous (roughly a million) nephrons. The nephron 44 is the smallest structural and functional kidney unit that filters nutrients and/or waste products that enter the blood stream into waste (urine) and re-absorbed nutrients (nutrients absorbed back into the blood). Blood flow enters the kidney via the afferent arterioles and passes into the glomerulus, which is encompassed by a Bowman's capsule. A significant portion of the filtration takes place in the Bowman's capsule, where the flow divides into two parts: filtered blood carrying macromolecules, large proteins, and red blood cells flow into blood vessels 46 (arterioles, capillaries, veins, etc.) and filtrate 48 (smaller molecules, ions, etc.) flows into other components of the nephron 44 (tubules, Henle's loop, ascending/descending limbs, collecting ducts, etc.). Solutes in the blood undergo a series of filtration (including secretion) and re-absorption processes, along a flow direction that is transverse to the blood flow.

The kidney model includes various equations that express transport dynamics at various spatial locations in the kidney and/or connected to the kidney. The equations describe transport of mass (e.g., solutes, ions, nutrients, wastes, etc.), fluid (e.g., filtrate, blood, etc.), heat, etc. in time, at or through different spatial locations. The described transport may be via different mechanisms (e.g., hydraulic, osmotic, advective, chemical, electrical, cotransport, etc.).

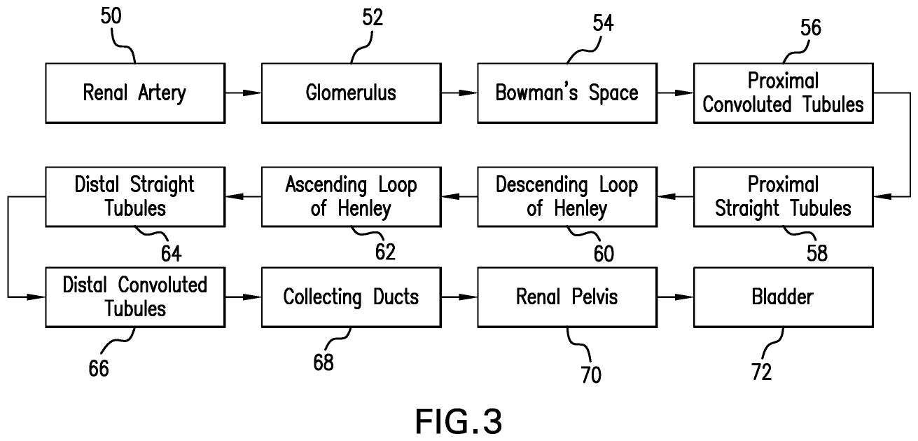

FIG. 3 depicts components of the kidney model, which simulates at least fluid and solute flow through various spatial locations with the inclusion of hydraulic and mass transport domains. In this example, the computer system 10 and/or other processing device simulates spatial locations in order of fluid flow through the kidney. In this example, fluid and solutes flow axially though the kidney, through various structures, and to the bladder. The kidney model may also simulate transverse flows (not shown in this example), which include fluid and solutes flow to the neighboring cells, intercellular spaces, and interstitial fluid.

It is noted that additional, fewer, or alternate transport domains than those shown can be embodied (e.g. electrical, chemical, etc.). It is also noted that the spatial locations may be more, fewer, or different than those shown (e.g. ureter, interstitial fluid, aorta, etc.).

In the example of FIG. 3, the transport of solutes, ions and fluid in time is expressed via differential equations. The arrows in FIG. 3 indicate the axial flow of fluid (flow of fluid and solutes 48) as a function of time and also show the order in which each spatial location is simulated. The spatial locations in this example include the renal artery 50, the glomerulus 52, Bowman's space 54, proximal convoluted tubules (PCT) 56, proximal straight tubules (PST) 58, the descending loop of Henley 60, the ascending loop of Henley 62, distal straight tubules (DST) 64, distal convoluted tubules (DCT) 66, collecting ducts 68, the renal pelvis 70 and the bladder 72. Each of these spatial locations are associated with a respective set of equations, such as differential equations, that can be solved to simulate fluid and mass flows.

Other spatial locations that are positioned transversally relative to the axial flow can also be simulated. Examples of such transverse spatial locations include epithelial cells, intercellular junctions, and interstitial fluid neighboring the spatial locations, which are shown in FIG. 3.

At each spatial location, continuity (e.g., mass balance or Kirchoff Current Law) and compatibility (e.g., Kirchoff Voltage Law) equations can be used, as well as energy transduction or constraint equations where needed. All these equations then collectively form the kidney model.

The equations can be differential or algebraic equations. As is customary, the number of equations has to equal the number of unknown variables so the system of equations can be solved and describe the transport dynamics.

The locations in FIG. 3 are not meant to be restrictive, and locations could be added or removed as needed, even to include spatial locations for other organs.

The kidney model can be used to simulate kidney function generically, simulate kidney function for a class of patients (i.e., patients having one or more common characteristics) and/or simulate kidney function for an individual patient. A model that is tuned to an individual patient is referred to herein as a “personalized model.”

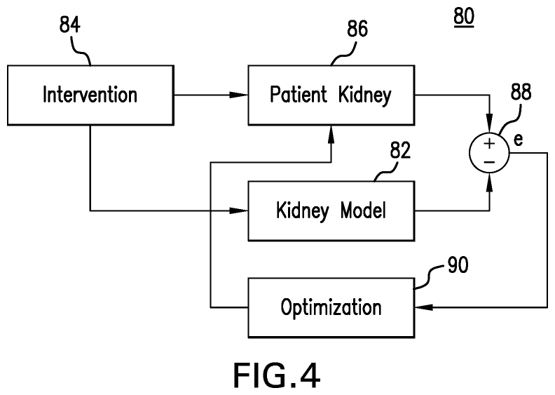

FIG. 4 illustrates aspects of a kidney evaluation method 80 that includes personalizing the kidney model for an individual patient. A model 82 is generated that generally captures the dynamics of the kidney and is built based on first principles, i.e. it is based on the physics of the physiology as understood by those skilled in the art. The model 82 at this point is an initial model that is (at least substantially) not a data-based model and hence there is no training to be done requiring data. Data can be used, however, but after the initial model has been developed and for the purpose of model validation. Once the model is personalized, the personalized model can be used for various purposes, such as running what-if clinical scenarios. The what-if scenarios can be run, e.g., following the Intervention to Kidney Model branch of FIG. 4.

The model 82 is then fine-tuned in order to personalize the model 82 for an individual patient. FIG. 4 illustrates an embodiment of the method 80 that includes personalization of the mathematical kidney model. Fine-tuning could include selecting or changing model inputs, initial conditions or initial patient health status (e.g., as an initial parameter value set), changing parameter values, applying interventions, and any other change. In one embodiment, the model 82 is fine-tuned by inputting patient data corresponding to an individual patient to the model 82 and calculating a response of the model 82 thereto.

“Patient data” generally refers to any data or information that corresponds to or is associated with an individual patient. Examples of patient data include vitals such as blood pressure and temperature, demographic information, interventions applied to the patient or applied as an input to the model, and other inputs that reflect characteristics of the patient.

In one embodiment, an intervention 84 (such as fluid bolus) is given to an actual kidney 86 of the individual patient and its digitized form (value) is given to the model 82. An intervention can be any exogenous factor, such as environmental, chemical, biological exposure, or traumatic event resulting in a change of the initial conditions (initial values of the variables or parameters) of the model 82. An intervention does not only have to be an exogenous factor, however. An intervention can also be an endogenous stimulus, such as firing of the SA (sino-atrial) node, or difference in initial conditions, or a disease. Fine-tuning of the model need not include interventions, however, inclusion of interventions as well as corresponding outputs (e.g., physiological measurements of the patient, or outputs from the model) can yield better parameter estimation results.

The method 80 includes measuring the response of the patient kidney 86 to the intervention 84. In addition, the intervention is applied digitally to the model 82 to generate a simulated or model response. The measured actual kidney response and the model response are output to a comparator 88 that calculates a difference (shown as an error e) therebetween. The error e is output to a processing module such as an optimization module 90 (e.g., as a part the evaluation module 28 or as a separate module) that outputs changes in parameters; these changes, when applied to kidney model 82 parameters, tune the model 82 to minimize the error e. The kidney model may be tuned to approach a minimum error e of zero or some other selected value. The kidney model 82 is thereby personalized for the individual patient.

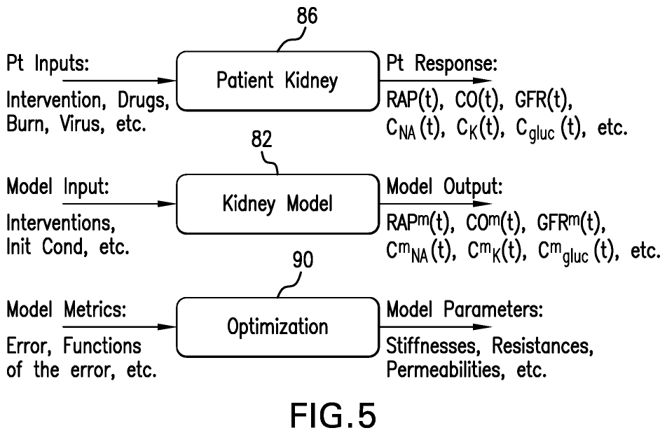

FIG. 5 illustrates examples of inputs and outputs of the different blocks of FIG. 4. The input to the patient kidney 86 (intervention) may include external stimuli (e.g. bacteria, virus, burn, and/or smoke inhalation) or therapeutic interventions (e.g., fluid including nutrients and/or drugs), and the output is the body's physiological responses. t indicates time. Examples of measurements of kidney response include arterial blood pressure (ABP) as a function of time (ABP(t)), cardiac output (CO) as a function of time (CO(t)), glomerular filtration rate (GFR) as a function of time (GFR(t)), and the concentrations of sodium, potassium, and glucose in time, among others.

Inputs to the model 82 are the initial conditions (i.e. the initial values of the variables that make the set of (differential) equations to run), as well as digitized versions of the interventions that are given to the patient. Outputs of the model 82 include modeled values of the patient response (the superscript m in the model outputs indicate “model”). Examples of such outputs, not exclusive, are shown in FIG. 5. The input to the optimization module 90 includes the error e between the patient response and the model response or a function of the error (e.g. error sum squares). The output of the optimization module 90 is a set of parameters (physical properties of the kidneys) that make the error e approach zero. In some embodiments, the model metrics can include fitness functions and the optimization module would attempt to maximize the fitness (rather than minimize the error).

As noted above, tuning the model 82 includes calculating an error or difference between the kidney response and the model response. As the initial model is configured to represent a healthy adult kidney (or a kidney representative of a certain patient-class based on demographics, comorbidities, environmental exposures and/or acute conditions), the initial model's response will differ from that of the real patient's kidneys, initially. The error from the output of the kidney model is compared to the real patient output (e.g., a type of patient data). In some embodiments, this patient data is subjected to filtering, scaling, normalizing, or other pre-processing procedures before they are input to the optimization module.

As an example, if the output is urine output (UO), then the model output is a value of the UO (computed as a summation of the urine outflow in time), and the real patient's UO is the urine collected over the same period of time. That error ideally would be zero, since then, the model would be outputting the same urine output as the patient. This error, however, can be minimized (to become zero or another minimum value) by fine tuning the kidney model (i.e. to change the coefficients in the equations of the kidney model). Other examples of outputs include the following: blood flow, fluid volume, or fluid/blood pressure in the renal artery, renal vein, systemic circulation, or glomerulus, concentrations of solutes (e.g. Na, K⁺, H⁺, Cl⁻, H₂CO₃, HCO₃⁻, glucose, protein, urea, etc.) in urine, concentrations of solutes (e.g. Na, K⁺, H⁺, Cl⁻, H₂CO₃, HCO₃⁻, glucose, protein, urea, etc.) in renal arterial or venous blood, and concentrations of solutes (e.g. Na, K⁺, H⁺, Cl⁻, H₂CO₃, HCO₃⁻, glucose, protein, urea, etc.) in systemic blood (plasma, serum, etc.), or functions of those variables thereof (e.g., pH).

The coefficients are changed according to an optimization algorithm executed by the evaluation module 90. The evaluation module 90 iteratively adjusts one or more parameters (e.g., coefficients) of the equations, or corresponding scaling factors, making up the model to reduce the error. The iterations may be performed continuously (e.g., as data becomes available), periodically or according to any selected schedule.

As such, and through iterations, the error e becomes zero as the coefficients are modified, and the model's output(s) start to converge toward the patient's output(s). Once the error reaches a target value (e.g., the error becomes zero or within a selected range from zero), the final set of model coefficients become the optimized parameter set. The coefficients can be modified until the error reaches zero or other target value, which can be any selected minimal value.

As noted above, parameters can be synonymous with coefficients and represent characteristics such as vessel and fluid properties, solute properties (e.g., type of solute), kidney tissue and tubules geometric and physical properties. For instance, parameters can include but are not limited to mass, length, diameter, resistance, compliance, inertance, feedback gains, and filtration, reflection, diffusion, frictional, and/or transporter coefficients. Parameters to be tuned can also be represented by corresponding scaling factors. Deviations of these properties from their initial (or healthy) values indicate different kidney diseases.

For instance, if during the course of a simulation, and while the optimization iterations try to make the error zero, the renal artery diameter (parameter) has converged (in iterations) to a value that is half of its initial (normal) value, then this would mean that most likely the physiological renal artery diameter, of the patient, is halfway blocked. The model then solves the resulting altered blood flow and pressure, likely causing (indicating) an abnormal condition inside the kidney. Since the kidney model is assumed to mimic the real kidney, then conclusions made on the model are assumed to be present in the patient's kidney. As such, the end result of the iterative optimization steps, which minimize the error, yields a parameter set that is indicative of kidney disease of the particular patient. We have, thus, “personalized” the kidney model to the individual patient's kidney. Hence, we can say that for the time period considered, the model is emulating the real kidney (of the patient), and as such any therapeutic simulations done on the model can be concluded for the patient—without touching the patient. One can then conclude that noninvasive, personalized medicine is being practiced on the patient for the time period considered. The relevancy of the personalized kidney model can be, for example, the time range between data acquisitions (e.g., a time between running the model using a preceding ICU data record and receiving the next available ICU data record).

This iterative process (measure, minimize error, obtain optimal parameter set, and simulate model (solve for model variables forward in time)) can be performed frequently, and even continuously, so that continual therapeutic simulations of a particular patient can be run as kidney health changes in time. Further, and without loss of generality, we can extend the definition of error e in FIG. 1 to include not only one time signal of one variable (flow, volume, pressure, etc.), but a plethora (vector) comprising time signals of a multitude of measured physiological variables (e.g., serum creatinine, arterial blood pressure, serum sodium (concentration), UO, etc.)

If we only know outputs or measured physiological variables (e.g. flow, volume, pressure, concentration), an intervention is not known, we do not have a physiological model, and/or we have a model that is not complete or rigorous, then we can perform (output-only) system identification and assume some intervention or disturbance. The intervention assumed can then be any signal, such as white noise, or can be a signal selected from intervention scenarios on patients from the same patient class. If, however, we know inputs or measured interventions given to (affecting) the patient as well as outputs (measured variables) of the patient, then we can perform input and output system identification (as described above).

In one embodiment, the systems described herein are configured to generate diagnostic and/or therapy recommendations, which can be tailored to an individual patient based on the personalized model.

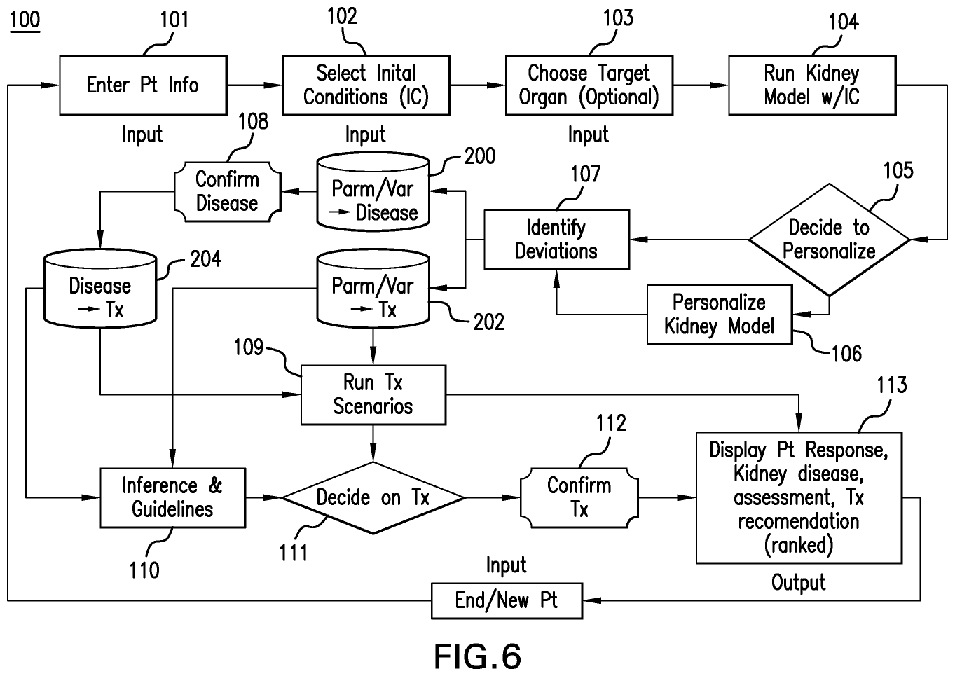

FIG. 6 is a block diagram showing aspects of a method 100 of evaluating kidney function and generating treatment recommendations, which can be performed by or with a computing or processing device such as the computer system 10. The method 100 includes a number of steps or stages represented by blocks 101-113. The method 100 may include all of the steps or stages in the order discussed, may include fewer than all of the steps or stages, or may include additional steps or stages not shown.

The blocks 101-113 belong to three categories, two of which are shown by titles written below the blocks. Input blocks 101, 102 and 103 pertain to data entry or selection of information that is needed in order to run the kidney model 82. Output block 113 represents all the necessary modules to display timely information of diagnosis and therapy recommendation for the individual patient at hand. The remaining blocks are algorithm or model blocks related to calculations using the kidney model 82.

At block 101, patient information including patient class data is acquired or entered (e.g., via a graphical user interface, data application programming interface, etc.), including demographic information (e.g. age, gender, race, height, weight, body mass index, etc.), which is used to retrieve the appropriate initial parameter set (set of initial coefficients for the model equations) of a patient class from a data repository of initial parameter and variable sets. Each set of parameters and variables is referred to as a “Parm/Var” set. Each Parm/Var set belongs to a patient class and patient classes can be distinguished by demographic and/or diagnostic (chronic, current) information. Patient information can also include chronic conditions or acute/current diagnoses that would further help to select the initial parameter set (e.g., patients with COPD would have high upper airway resistance).

At block 102, initial conditions are selected, which include initial (or steady-state) values of the parameters and the variables. In the case of initial conditions for variables, patient information entered or acquired can also include initial values of measured variables (e.g. vitals, labs, etc.) or interventions (e.g. fluids, meds, dialysis, etc.) done to the patient thus far. The initial conditions enable the running of an initial version of the kidney model 82 (an initial model).

At block 103, the method optionally includes selection of a target organ. For example, patient data (e.g. vitals, labs, etc.) is used to find the organ system, organ sub-system, or organ-organ pathway that is the target, or primary focus, of the patient's current condition. This stage can result in a reduced model, or reduced set of equations relative to the initial model, to be used for optimization or error minimization 90.

If the target organ is not identified at this stage, the processing device will instead perform a leave-one-out procedure whereby one organ system, sub-system, or organ-organ pathway is omitted from the model at a time, error between measured and model computed variables is computed, error minimization 90 is performed, and the minimal error is stored; after all the leave-one-out trials, the model simulation with the minimal error, or best fit to the real patient's measured variables, is selected as the reduced model to be used for (future) optimization 106. The leave-one-out procedure can be extended to leaving one or more organs, organ sub-systems, or organ-organ pathways out at a time. The chosen target organ can also be thought of as the chosen culprit organ. For this is the organ whose corresponding subsystem will be the optimization focus. In a single organ model, the chosen target organ can be thought of as one or more important spatial locations. Alternatively, the organ can be identified via an Artificial Intelligence procedure (e.g., inference system, Neural Network type approach, etc.)

At block 104, the model 82 is run with the initial conditions. At this point, the model 82 is considered an initial model. The initial model includes equations for each spatial location of a simulated kidney. At block 105, it is determined whether to personalize the model. This determination may be made automatically, or an option may be provided to a user (e.g., a physician or nurse) via a suitable display or graphical user interface. The decision to personalize hinges on what we are seeking: the response of the individual patient (personalized) or the response of a patient class (group).

At block 106, the model 82 is personalized for a selected patient class and/or for an individual patient. As discussed above with reference to FIG. 4, this stage involves the response of the model 82 that can be one or more variables in time to be then compared to the measured variables from the real patient creating an error signal (e.g., the error e of FIG. 4) that is used to optimize the model 82 for the patient class or individual patient.

At block 107, deviations of model outputs or responses from normal (healthy) kidney responses can be calculated. Deviations from normal can be differences in variables (or parameters) compared with those of normal (healthy) patients, those of the same patient class, or those of the same patient at an earlier point in time. These deviations can be determined via statistics or fuzzy or Bayesian inference, or the like, on large datasets or many prior model simulations. The processing device may then consult an appropriate database, look-up table or other source of information to identify a disease based on the deviations.

Blocks 108-113 represent aspects of the method 100 that include using knowledge databases for model driven disease identification and/or therapy recommendations. The knowledge databases (including, e.g., look-up tables or other structures) may include databases that associate deviations of model outputs (deviations in the coefficients (parameters) or model responses (variables)) with various diseases, and/or databases that associate deviations and/or model parameters with specific therapies. These databases can be pre-existing or generated or put together from existing bodies of knowledge or a combination thereof. Although the databases and the method 100 are discussed in the context of kidneys, the databases and/or the method 100 may be used in conjunction with other organ systems.

One database shown in FIG. 6 is a database 200 that stores parameter and/or variable values and associates different values (or value ranges) with respective diseases. The database is also referred to as a Parm/Var→Disease database 200. In one embodiment, the Parm/Var→Disease database 200 maps ranges of parameters and variables (or their deviations from normal) to specific renal diseases, for a specific patient class. This body of knowledge can be in the form of rules or guidelines. For instance, if the renal arterial blood pressure is high and urine output is low and urine concentration of calcium is high then hypercalcemia, say, could be the most likely renal disease diagnostic culprit. In its simplest form, this database would include at least three of the following: a variable or parameter (e.g. blood pressure), a direction (e.g. low, high, very low, very high), a threshold (e.g. <60, <=90), a normal or reference range (e.g. 60-120), and a disease (e.g. hypertension).

Another database is a database 204 that stores information related to diseases and associated therapies. The database 204 is also referred to as a Disease→Tx database 204, which maps a given disease (or condition) to therapies. A “disease” as described herein relates to any disease, condition or other type of sub-optimal function.

The Disease→Tx database 204 can be similar to pharmacological databases where drugs are indicated for specific diseases or conditions. For instance, if a patient has a restrictive lung disease, then a drug that is specific to that disease in that patient's class would then be assigned (e.g. bronchodilator), so as to reduce the bicarbonate buildup in the kidneys. In its simplest form, this database would include a disease or condition (e.g. restrictive lung disease) and at least two of a therapy type (e.g. ACE inhibitors), a therapy level (e.g. high dose or low dose), a therapy dose (e.g. 20 mg), a therapy rate (e.g. 1.5 mg/mL), a therapy duration (e.g. 10 min, 10 hr), and a therapy frequency (e.g. 2×/day, one time).

An alternate pathway involves another database referred to as a Parm/Var→Tx database 202 that maps the ranges of parameters and variables (or their deviations from normal) directly to therapies. This again can be similar to pharmacological databases, where drugs are indicated for correcting (e.g. restoring to normal for that patient or patient class) certain observed conditions (e.g. variable changes) or biological, immunological, or physical conditions (e.g. parameter changes). For instance, if a patient has high blood pressure but normal cardiac output, an ACE inhibitor can be used to lower system vascular resistance with little effect on cardiac output. In its simplest form, the Parm/Var→Tx database 202 can include at least two of a variable or parameter (e.g. blood pressure), a direction (e.g. low, high, very low, very high, above, below), a threshold (e.g. >160, >=155), and a normal or reference range (e.g. 90-130), and at least two of a therapy type (e.g. ACE inhibitors), a therapy level (e.g. high dose or low dose), a therapy dose (e.g. 20 mg), a therapy rate (e.g. 1.5 mg/mL), a therapy duration (e.g. 10 min, 10 hr), and a therapy frequency (e.g. 2×/day, one time).

At block 108, the processing device, identifies a disease by correlating a deviation from normal and/or coefficient values (derived from optimizing or personalizing the model 82) with a specific disease. This stage may include consulting rules or guidelines to determine an appropriateness of the identified disease prior to moving forward to the next step or making a renal health assessment of that disease.

At block 109, from either the Var/Parm→Tx database 202 or the Disease Tx database 204, are consulted to determine a diagnosis and/or therapy recommendation. For example, inference logic, internal what-if intervention scenarios checks (e.g. simulations on the model of the therapy to see if it indeed brings the deviated parameter or variable back into a normal range for that patient or patient class), or guidelines can be invoked or consulted next to ensure the appropriateness of the diagnosis or therapy recommendation (block 110). For example, at block 110, specific logic and conditionals are invoked to ensure suggested therapies are those normally presented according to the current state of the art of physician knowledge.

Also at block 110, various intervention scenarios can be applied to the model 82 to determine the appropriateness of the therapy recommendation. For example, once the model 82 is tuned to an individual patient, a number of therapy scenarios (e.g., based on therapy recommendations from the knowledge databases), are applied to the personalized model to assess the appropriateness and/or effectiveness of such therapies for that specific patient. Based on this rich individualized diagnostic and therapeutic information, it can then be decided which therapies to recommend (block 111)

Additionally, or alternatively, the results from the scenarios (responses or variables (or parameters) in time can be shown to a user (e.g., a physician) with the corresponding therapy, and the user can decide upon the diagnostic or therapeutic course of action based on the therapy that produces the most desirable response.

At block 113, evaluation, diagnostic and/or therapeutic information is displayed to a user. Various types of information can be displayed, such as model response, patient response, variable and parameter data, potential diseases diagnosed, recommended treatments and others. For example, the personalized model can be displayed as a graph that plots measurements from patients and estimates from model. Possible diseases can be displayed (with, e.g., causative variables and parameters, and/or deviations from normal/baseline). The system 10 can display one or multiple treatment options (e.g., “you may want to consider one of these . . . ” or “based on Merck, etc., we suggest . . . a, b, c, or d” where a, b, c, and d refer to different treatment options with one or more steps).

Other information that can displayed includes hypothetical responses to intervention scenarios. For example, patient response given different interventions (varied med, dose, duration, etc.) can be displayed, as well as advice regarding which disease to identify based on shown responses.

Additionally, or alternatively, the results from the scenarios (responses or variables (or parameters) in time) can be shown to the user with the corresponding therapy, and the results can be ranked based on those which most closely reach a desired (or set by user/physician) therapy target. These therapy targets can be set by: 1) those that restore variables or parameters to normal or reference ranges, 2) those that restore a patient to his/her normal (or that of the patient class), 3) those that restore variables or parameters of the patient in a desirable time horizon (e.g. quickly/short time, slowly, etc.), and/or therapies identified using options 1), 2) or 3) while avoiding harmful conditions (e.g. deviating other variables or parameters from their normal ranges). In addition, therapy targets can be shown that do 1) or 2) or 3) while avoiding the need for additional intervention or therapy (e.g. patient requires additional therapy to restore other variables or parameters).

Additionally, or alternatively, the results from the scenarios (variables in time) can be ranked based on those which minimize the summation of one or more of the therapy objective functions. The therapy objective functions would be those that describe one or more functions (e.g., filtration, metabolism, oxygenation, circulation, acidity, etc.) of one or more organs constituting health or homeostasis.

In another embodiment, the method 100 can omit the knowledge bases shown in FIG. 6 and proceed directly from block 106 to block 109. In this embodiment, once the model 82 is tuned to an individual patient, the model 82 can be used by a user (e.g. physician or other care provider) for specific on-demand intervention scenarios where the user could test the therapy on the model 82 and see the response; if the response is desirable, the user can then make decisions regarding appropriate course of action. This differs from the former embodiment in that the scenarios are user initiated and defined. In yet another embodiment, the therapy recommendation logic is connected to a therapy ordering or inducing/delivering system or device, where a health care provider can authorize such a dispense so it then can be processed.

The method 100 and other embodiments of methods described herein may include various actions performed based on the model. For example, the model 82 can be used for educational purposes in clinical patient simulation laboratories, in simulation model-based mannequins, classrooms, etc., by running scenarios and seeing the behavior of different transports for various applications. What-if diagnostic scenarios can be run, for instance, by changing some renal tissue properties, in essence simulating kidney damage (illness) and seeing the ensuing fluid and solutes' transport (filtration) responses. What-if therapeutic scenarios can also be run giving the model a simulated therapeutic intervention and letting the model present the resulting responses.

In another embodiment, the methods can include designing exogenous devices that deal with the kidneys, such as dialysis machines for renal replacement therapies. For example, fine tuning machine-kidney interactions, and hence optimizing the filtration operation, can be done by simulating the whole operation, i.e. the patient (the kidney dynamic model) and the machine interacting with this virtual patient or personalized kidney model, and by running different intervention (or kidney damage) scenarios. This allows one to find the limit of operation of the machine (safe vs. harmful regions of operation) and the response of the kidney due to different machine settings and for different kidney diseases. The knowledge gained would allow for better design of the machine. Other potential uses in this space could include the following: identifying optimal flow rates for preventing adverse patient events and/or reducing dialysis time; identifying optimal type or concentration of dialysate fluid (or solutes in it) to effectively and efficiently administer/remove/replace solutes or waste products (at varied concentration levels) from the blood. Another example of this embodiment is in the designing of kidney-smart, safe medication infusion pumps, which optimize drug/fluid delivery flow rate, volume, and duration in a manner to minimize harm to the kidneys (e.g. tubular damage or injury).

In another embodiment, the methods are used to administer/practice personalized medicine. Potential uses include the following: predicting disease onset or trajectory via expressions of variables (or parameters) in time; providing end-users with the ability to perform what-if clinical intervention (or disease) scenarios on the model 82 (thereby preventing trial-and-error on the real patient, enabling better decision-making). Further uses include performing in-silico intervention (or disease) scenarios via a model of the physician, guidelines, or other mechanism and providing a therapy recommendation. The responses of all scenarios via disease trajectory (expression of variables in time) can be used as a mechanism for clinical decisions, for example, by providing advice on which of the scenarios provides better outcome, and/or providing advice on which scenarios meet a pre-defined end-user target.

In one embodiment, systems and methods described herein are configured to forecast potential kidney diseases or conditions (or generally forecast kidney response) by simulating a kidney response based on a personalized model (e.g., the model 82) at one or more selected future times. For example, the model can be run (with or without simulated interventions, actual interventions and actual kidney responses to such interventions, and/or what-if scenarios) to forecast patient variables in time. Such forecasted patient variables can be used for diagnostic support information. The forecasted patient variables may be correlated or associated with risk information, such as a risk of a patient developing a kidney disease or condition (e.g., using one or more databases such as the databases 200, 202 and/or 204).

Embodiments described herein can be used for various applications. In one embodiment, simulations using a personalized kidney model are performed (e.g., continuously or periodically) to monitor the health of a patient kidney and/or monitor the status of a disease or condition. For instance, the glomerular filtration rate (GFR; defined as how much blood passes through the glomeruli as a function of time) and/or creatinine clearance (C_{Cr}; defined as how much blood plasma volume is cleared of Creatinine per unit time) can be non-invasively and continuously estimated (assessed) using embodiments described herein. This is simply not possible in current day medicine. GFR is an indication of kidney health, i.e. how well the kidneys filter out waste from the blood. GFR is currently not measured, but is instead calculated (estimated) via formulae using blood tests and termed eGFR. Markers, radioactive tracers, and invasive means are sometimes used as attempts to estimate this quantity; however, each attempt comes with its own cost in terms of patient harm or discomfort, expense, and latency of lab results.

C_{Cr}, can also be used to assess GFR. It is currently measured via collecting urine over a 24-hour period with frequent comparative blood samples.

Embodiments described herein address the above problems by providing for non-invasive monitoring and estimation of variables without requiring invasive techniques currently used. In addition to the above examples, other renal variables can be continuously and non-invasively estimated.

As noted above, these renal variables indicate kidney health. They can also be calculated in future time in order to provide forecasting of renal health, since the time trajectory for each can easily be calculated. These variables in time trajectories can pertain to an individual patient (and not a generic one) if personalized parameters are provided (and previously computed as described above.) This forecasting of the variables can be done in the presence or absence of therapeutic scenarios.

As noted above, the system can be used to generate one or more therapy recommendations based on a determination using the model that a patient has or is at risk of developing a disease or condition. Therapy recommendations may be performed, once a physiological model has been personalized, by forecasting kidney health and/or disease, and providing a therapy recommendation (or multiple recommendations) based on the forecast.

Kidney (or other organ) health, in one embodiment, is forecasted using a personalized model (e.g., the model 82). The forecast may be a prediction that a patient kidney is generally healthy, exhibits some form of sub-optimal performance and/or has or is at risk of developing a disease or condition. The model is solved forward in time; the outputs (response variables in time) indicate health/disease trajectory and provide a time-based risk indication or prediction of time. Based on the indications, a therapy recommendation can then be provided.

Therapy recommendations can be provided either directly or indirectly through use of intervention scenarios initiated by a user or automatically. Each intervention scenario can be applied by inputting a digital representation of an intervention or interventions, and generating a model response representing a patient (or patient class) response to the intervention. The model response is evaluated to determine whether an intervention has a positive or desired effect on the model.

In one embodiment, therapy recommendations are generated directly by applying intervention scenarios to the model by testing one or more interventions, and obtaining model response in the form of changes in one or more variables over time. The model responses are optionally displayed to a user (e.g., a physician or researcher). One or more therapeutic interventions (e.g., therapy rules, guidelines, or custom/user-defined intervention targets) is/are applied to the patient response variables (or risk prediction in time). One or more interventions are recommended that are determined to have a desired or positive effect on the model, e.g., meet the rules, guidelines, or defined intervention targets. A recommended intervention may be the applied intervention that achieves a desired target (e.g. look at levels of glucose in blood in time and ensure the intervention can maintain tight glucose control between 140-180 mg/dL), or that which minimizes disease risk prediction (e.g. lowest glomerular nephritis risk in time). If none of the interventions are suitable, a recommendation not to use the tested intervention(s) may be displayed.

In one embodiment, therapy recommendations are generated indirectly by applying intervention scenarios to the model by testing one or more interventions, and obtaining variable values as a function of time due to the model response. A set of one or more variable values are generated for each of a plurality of different intervention scenarios, and may be displayed to a user. The sets of variable values from each intervention scenario are provided for comparison and the user compares results (patient response in time from different interventions) and selects the desirable patient response (e.g. increase vascular fluid volume while maintaining cell and tissue fluid volume; increase vascular pressure quickly), thus indirectly choosing the intervention that yielded that patient response.

The following description includes examples of intervention scenarios that can be applied to a model:

1. Administer normal saline to a patient at different infusion rates and over varied durations. Select the intervention (from response) that keeps blood pressure highest (or prevents blood pressure from dropping below 65, for example), while also not causing metabolic acidosis (e.g. pH<7.35).

2. Administer normal saline and Ringer's solutions at different doses, infusion rates, and durations. Observe the blood pressure in time and sodium concentration in the intracellular or extracellular fluid (intercellular junctions, interstitial fluid, etc.). Select the intervention that prevents sodium retention and need for additional fluid bolus.

3. Administer loop diruetics at different dose and infusion rate and compare to administration of RRT at different dialysate flow rates. Select the intervention that achieves desired filtration and clearance while avoiding hemodynamic events (drop in blood pressure).

4. Administer cisplatin at different doses, infusion rates, and over varied durations. Observe the glomerular filtration rate in time and the glucose and protein concentrations in the urine in time. Select the one that maintains adequate GFR and minimizes amount of glucose and protein in the urine.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments of the invention, the practical application or technical improvement over technologies found in the marketplace, or to enable others of ordinary skill in the art to understand the embodiments described herein.

What is claimed is:

1. A system for evaluating a kidney, the system comprising:
a processing device including:
an input module configured to acquire patient information, the patient information including at least one of demographic data, diagnostic data, physiological data and intervention data;
an evaluation module configured to perform:
inputting patient class data to an initial kidney model, the initial kidney model configured to simulate a physiological response of a kidney, the initial kidney model configured to simulate fluid and solute transport through one or more spatial locations of the kidney;
inputting patient data corresponding to an individual patient and calculating a model response; and
adjusting at least one parameter of the initial kidney model based on a comparison of the patient data and the model response to personalize the initial kidney model for the individual patient.

2. The system of claim 1, wherein each of the initial kidney model and the personalized model include a plurality of mathematical equations, each of the plurality of mathematical equations representing transport phenomena of fluid and mass conservation through one or more spatial locations of a kidney.

3. The system of claim 1, wherein adjusting the at least one parameter includes comparing the patient data to the model response and calculating an error therebetween, and adjusting the at least one parameter to reduce the error.

4. The system of claim 3, wherein adjusting the at least one parameter includes iteratively adjusting the at least one parameter until the error or a change in the error is at or below a selected minimum value.

5. The system of claim 1, wherein the evaluation module is configured to continuously or periodically monitor the actual kidney based on resulting parameters or variables of the personalized model.

6. The system of claim 3, wherein the evaluation module is configured to diagnose a condition or disease of the actual kidney based on a difference between a first value of the parameter and an adjusted value of the parameter.

7. The system of claim 3, further comprising a database configured to store kidney disease information, the kidney disease information including one or more values of variables and/or parameters corresponding to known diseases.

8. The system of claim 7, wherein the evaluation module is configured to diagnose a kidney disease of the individual patient by comparing model response variables or the adjusted at least one parameter to the kidney disease information.

9. The system of claim 1, wherein the evaluation module is further configured to forecast a value of the at least one variable or parameter by simulating a kidney response based on the personalized model at one or more selected future times.

10. The system of claim 9, wherein the forecast includes a prediction of a risk of a kidney condition or disease.

11. The system of claim 1, wherein the evaluation module is further configured to generate a therapy recommendation and present the therapy recommendation to a user based on the model response data.

12. The system of claim 11, wherein the model is the personalized model and the evaluation module is configured to generate the therapy recommendation by applying one or more interventions to the personalized model, and calculating a model response to the one or more interventions.

13. The system of claim 12, wherein the one or more interventions include one or more therapeutic interventions, and the evaluation module is configured evaluate an effect on the personalized model of the one or more therapeutic interventions.

14. The system of claim 13, wherein the effect is evaluated by minimizing or maximizing objective functions describing organ health.

15. The system of claim 1, wherein inputting the patient data includes acquiring kidney response data indicative of a response of the patient's actual kidney to an intervention applied to the actual kidney, the intervention including at least one of an external input and a disturbance.

16. The system of claim 15, wherein inputting the patient data further includes digitally applying the intervention to the model, the model response including a response of the model to the digital intervention, and the adjusting is based on a comparison of the patient response and the response of the model to the digital intervention.

17. A method of evaluating a kidney, the method comprising:
acquiring patient information at an input module, the patient information including at least one of demographic data, diagnostic data, physiological data and intervention data;
inputting patient class data to an initial kidney model, the initial kidney model configured to simulate physiological responses of a kidney, the initial kidney model configured to simulate fluid and solute transport through one or more spatial locations of the kidney;
inputting patient data corresponding to an individual patient and calculating a model response; and
adjusting at least one parameter of the initial kidney model based on a comparison of the patient data and the model response to personalize the initial kidney model for the individual patient.

18. The method of claim 17, wherein each of the initial kidney model and the personalized model include a plurality of mathematical equations, each of the plurality of mathematical equations representing transport phenomena of fluid and mass conservation through one or more spatial locations of a kidney.

19. The method of claim 17, wherein adjusting the at least one parameter includes comparing the patient data to the model response and calculating an error therebetween, and adjusting the at least one parameter to reduce the error.

20. The method of claim 19, wherein adjusting the at least one parameter includes iteratively adjusting the at least one parameter until the error or a change in the error is at or below a selected minimum value.

21. The method of claim 17, further comprising continuously or periodically monitoring the actual kidney based on the resulting variables or parameters of the personalized model.

22. The method of claim 19, further comprising diagnosing a condition or disease of the actual kidney based on a difference between a first value of the parameter and an adjusted value of the parameter.

23. The method of claim 19, wherein the evaluation module is in communication with a database configured to store kidney disease information, the kidney disease information including one or more values of variables and/or parameters correlated to known diseases.

24. The method of claim 23, further comprising diagnosing a kidney disease of the individual patient by comparing model response variables or the adjusted at least one parameter to the kidney disease information.

25. The method of claim 17, further comprising forecasting a value of the at least one variable or parameter by simulating a kidney response based on the personalized model at one or more selected future times.

26. The method of claim 25, wherein the forecasting includes generating a prediction of a risk of a kidney condition or disease.

27. The method of claim 17, further comprising generating a therapy recommendation and presenting the therapy recommendation to a user based on the model response data.

28. The method of claim 27, wherein the model is the personalized model and generating the therapy recommendation includes applying one or more interventions to the personalized model, and calculating a model response to the one or more interventions.

29. The method of claim 28, wherein the one or more interventions include one or more therapeutic interventions, and generating the therapy recommendation includes evaluating an effect on the personalized model of the one or more therapeutic interventions.
